(19) United States
(12) Patent Application Publication
(10) Pub. No.: US 2004/0120974 A1
(43) Pub. Date: Jun. 24, 2004

(54) Powder concentrates, methods of preparing the same, methods of using the same, and emulsified personal care products prepared from the same

(75) Inventor: Bruce W. Gesslein, Brick, NJ (US)

Correspondence Address: OBLON, SPIVAK, MCCLELLAND, MAIER & NEUSTADT, P.C., 1940 DUKE STREET, ALEXANDRIA, VA, 22314, US

(73) Assignee: AJINOMOTO USA, INC., Paramus, NJ

(21) Appl. No.: 10/326,613

(22) Filed: Dec. 23, 2002

(51) Int. Cl. A61K 7/38; A61K 7/00

(52) U.S. Cl. 424/401; 424/68

(57) ABSTRACT

The present invention relates to a method of preparing easily diluted powder personal care concentrates. The dilutions of these concentrates form useful, topical personal care products meant to be applied to the skin and the hair. The concentrate may also contain “active” substances that facilitate the function of the final diluted product. Additionally, the powder form of the concentrate obviates the need for anti-microbial preservation and facilitates easy transport.

### BACKGROUND OF THE INVENTION

1. Field of the Invention

The present invention relates to novel powder concentrates, which are useful for preparing emulsified personal care products. The present invention further relates to novel methods of preparing such concentrated powders and methods of using such powder concentrates. The present invention also relates to emulsified personal care products prepared from such powder concentrates.

2. Discussion of the Background

Personal care products are produced in a number of forms, many of which are emulsified lipid or lipid-soluble material and water or water-soluble material systems. These products may be moisturizers, creams, lotions, foundation make-ups, cleansers, skin clarifiers, sunscreens, skin and hair conditioners, hair styling products, anti-perspirants and deodorants, and the like.

Emulsion concentrates have been formed in the past by reducing the water content of a personal care product by fifty or sixty percent of its original content. These concentrates were useful, but required preservation against microbial attack, did not conveniently allow for single dose packaging, and often were physically unstable requiring the mixing of the concentrate before use.

Thus, there remains a need for personal care products which may be easily prepared from individual-sized containers or packages. There also remains a need for concentrated powders which may be easily diluted to form an emulsified personal care product, but are re more convenient to store than already prepared emulsified personal care products.

### SUMMARY OF THE INVENTION

Accordingly, it is one object of the present invention to provide novel concentrated powders which can be easily diluted to afford an emulsified personal care product.

It is another object of the present invention to provide novel methods of preparing such a powder concentrate.

It is another object of the present invention to provide novel methods of using such a powder concentrate. It is another object of the present invention to provide novel personal care products prepared from such a powder concentrate.

These and other objects, which will become apparent during the following detailed description, have been achieved by the present inventors' discovery that a powder concentrate, which comprises:

(a) a solid emollient or lipid; and

(b) a solid emulsifier,

may be easily diluted with an aqueous liquid to obtain an emulsified personal care product.

Thus, the basis of the present invention is to use powdered emollients/lipids and powdered emulsifiers. To this base a number of materials and active substances may be added to produce an end dilution with specific effects.

### DETAILED DESCRIPTION OF THE PREFERRED EMBODIMENTS

Thus, in a first embodiment, the present invention provides novel powder concentrates, which comprises:

(a) a solid emollient or lipid; and

(b) a solid emulsifier.

In the context of the present invention, the solid emollient or lipid may be any conventional solid emollient or lipid. Thus, the solid emollient or lipid may be a long chain, C₁₆-C₄₀, preferably C₁₈-C₃₀, alcohol; a long chain, C₁₂-C₃₀, mono-, di-, or triglyceride; an ethylene glycol diester of a long chain, C₁₆-C₃₀, fatty acid; an ester of a long chain, C₁₄-C₃₀, alcohol with a long chain, C₁₄-C₃₀, fatty acid; a salt (e.g., sodium, potassium, ammonium, etc.) of a long chain, C18-C₃₀, fatty acid; a C₁₂-C₃₀ fatty acid substituted amino acid; or a mixture thereof. Specific examples of the solid emollient or lipid include stearyl alcohol, cetearyl alcohol (a mixture of C₁₆-C₁₈ straight chain saturated fatty alcohols), cetyl alcohol, behenyl alcohol, glyceryl mono-, di, or tristearate, ethylene glycol distearate, cetyl palmitate, myristyl myristate, lauroyl lysine, aluminum stearate, zinc stearate, and mixtures thereof. Preferably, the solid emollient or lipid is cetyl palmitate, myristyl myristate, behenyl alcohol, lauroyl lysine, or a mixture thereof. Most preferably, the solid emollient or lipid is lauroyl lysine, myristyl myristate, cetyl palmitate, or a mixture thereof.

The solid emollient or lipid may be any emollient or lipid which has a solidification point greater than 35° C., preferably greater than 40° C., more preferably greater than 45°. Alternatively, the solid emollient or lipid may be one which is normally liquid at ambient temperatures, i.e., at about 20° C., but which has been rendered in a solid form by mixing with a solid such as urea or sodium carbonate.

The solid emollient or lipid will typically be present in the powder concentrate in an amount of 10 to 90% by weight, preferably 30 to 70% by weight, more preferably 40 to 60% by weight, based on the total weight of (a) the solid emollient or lipid and (b) the solid emulsifier. When the powder concentrate contains additional components, as described below, the solid emollient or lipid will typically be present in the powder concentrate in an amount of 7 to 85% by weight, preferably 20 to 65% by weight, more preferably 25 to 40% by weight, based on the total weight of powder concentrate.

Preferably, the solid emollient or lipid has a particle size such that its longest axis is less than 100 micron in length, more preferably less than 70 microns in length, and even more preferably less than 40 microns in length.

In the context of the present invention, the solid emulsifier may be any conventional solid emulsifier. Preferred classes of solid emulsifiers include salts (e.g., sodium, potassium, ammonium, etc.) of long chain, C₁₂₋₃₀, fatty alcohol esters of sulfates; mono-, di-, and triesters of long chain, C₁₂₋₃₀, fatty acids with sucrose; salts (e.g., sodium, potassium, ammonium, etc.) of long chain, C₁₂₋₃₀, fatty acids; salts (e.g., sodium, potassium, ammonium, etc.) of long chain, C₁₂₋₃₀, fatty acid esters and amides with glutamate; and mixtures thereof. Specific examples of the solid emulsifier include sodium lauryl sulfate, sodium cetearyl (a mixture of C₁₆-C₁₈ straight chain saturated fatty alcohol groups) sulfate, sodium cetyl sulfate, sucrose stearate, sucrose distearate, sodium stearate, sodium tallowate, sodium hydrogenated tallowoyl glutamate, sodium stearoyl glutamate, disodium stearoyl glutamate, sodium lauroyl glutamate, sodium myristoyl glutamate, sodium cocoyl glutamate, potassium lauroyl glutamate, potassium myrsitoyl glutamate, and admixtures thereof. Preferably, the solid emulsifier is sodium hydrogenated tallowoyl glutamate, sodium stearoyl glutamate, sodium lauroyl glutamate, sodium myristoyl glutamate, potassium lauroyl glutamate, potassium myristoyl glutamate, and admixtures thereof. Most preferably, the solid emulsifier is sodium hydrogenated tallowoyl glutamate, sodium stearoyl glutamate, or a mixture thereof.

The solid emulsifier may be any emulsifier which has a solidification point greater than 35° C., preferably greater than 40° C., more preferably greater than 45°. Alternatively, the solid emollient or lipid may be one which is normally liquid at ambient temperatures, i.e., at about 20° C., but which has been rendered in a solid form by mixing with a solid such as urea or sodium carbonate.

The solid emulsifier will typically be present in the powder concentrate in an amount of 10 to 90% by weight, preferably 30 to 70% by weight, more preferably 40 to 60% by weight, based on the total weight of (a) the solid emollient or lipid and (b) the solid emulsifier. When the powder concentrate contains additional components, as described below, the solid emulsifier will typically be present in the powder concentrate in an amount of about 7 to 85% by weight, preferably about 20 to 65% by weight, more preferably about 25 to 40% by weight, based on the total weight of powder concentrate.

The particle size of the emulsifier should be of a sufficiently small size to facilitate ease of dispersion in room temperature (20° C.) water by normal mechanical means.

The present powder concentrate may further comprise one or more additional active and functional materials may be added to produce personal care products of a number of types. These active and/or functional materials may include any or all, but are not limited to sunscreens, antiperspirants, moisturizers, humectants (e.g., trehalose), pigments, dyes, fragrance compounds, anti-bacterial agents, conditioning agents, vitamins, minerals, botanical extracts, exfoliants, protectants, calmatives, topical medicaments, and clarifying materials such as alpha hydroxy acids, beta hydroxy acids, enzymes, thickeners (e.g., acrylate/alkylacrylate cross polymers), and the like.

The exact amount of these additional active and/or functional agents included in the powder concentrate will depend on the final intended use of the powder concentrate. However, the total amount of active and/or functional agents will typically be from 5 to 50% by weight, preferably from 10 to 35% by weight, more preferably from 15 to 30% by weight, based on the total weight of (a) the solid emollient or lipid and (b) the solid emulsifier.

The present powder concentrates may be packaged in bulk, i.e., in amounts which are greater than the amount used to prepare a single application of the resulting emulsified personal care product. In a preferred embodiment, the present powder concentrate is packaged in an amount which corresponds to that use to prepare a single application of the resulting personal care product. In this case, the package will typically contain 0.5 to 50 grams, preferably 1 to 40 grams, more preferably 2 to 30 grams of the powder concentrate.

The present powder concentrate may be packaged in any suitable packaging material. Examples of suitable packaging include foil or plastic packs.

When it is intended to dilute the powder concentrate in a container other than the powder concentrate packaging, then the packaging need be no larger than required to contain the powder concentrate. Alternatively, when it is intended that the powder concentrate be diluted in the original package, then the package should be large enough to contain the powder concentrate and the aqueous liquid used for dilution. In this case, the package will typically have a capacity which is from 2 to 100 times larger, preferably from 10 to 50 times larger, than required to contain the powder concentrate alone. To save on storage space and shipping costs, it may be preferred to construct the packaging of a collapsible material, such as a flexible plastic, paper (such as a paper packet or paper straw with closed ends), or foil. It may also be preferred to seal the package under a vacuum.

In a second embodiment, the present invention provides a method for preparing a powder concentrate, said method comprising:

(1) Mixing (a) a Solid Emollient or Lipid and (b) a Solid Emulsifier

Thus, the present powder concentrates may be prepared by mixing (a) the solid emollient or lipid and (b) the solid emulsifier in the appropriate amounts. Any suitable means for mixing may be used for preparing the present powder concentrate. For example, the present powder concentrate may be prepared by mixing (a) the solid emollient or lipid and (b) the solid emulsifier in a drum mixing, a ball mill, a fluidized bed mixer, micro-pulverizer, etc. When present, the active and/or functional agents may be added during the mixing of (a) the solid emollient or lipid and (b) the solid emulsifier or may be mixed into the powder after the mixing of (a) the solid emollient or lipid and (b) the solid emulsifier has been completed.

After the mixing is complete, the powder concentrate may be packaged as described above.

In a third embodiment, the present invention provides a method for preparing an emulsion, preferably an emulsified personal care product, said method comprising:

(1) mixing:

(a) an aqueous liquid, and

(b) a powder, said powder comprising:

(1) a solid emollient or a solid lipid; and

(2) a solid emulsifier.

The aqueous liquid may be water or an alcohol-water mixture. In a preferred embodiment, the aqueous liquid is water.

Thus, the present method for preparing an emulsion may be carried out by simply mixing water with the powder concentrate described above. Typically, the water is used in an amount which is 1 to 90, preferably 2 to 80, more preferably 3 to 70, times the weight of the powder concentrate.

The mixing may be carried out in any number of ways. In a simple embodiment, the powder concentrate may be placed in the palm of the hand, along with a suitable amount of water, and the emulsion formed by rubbing the hands together. Alternatively, the emulsion may be formed by

Other features of the invention will become apparent in the course of the following descriptions of exemplary embodiments, which are given for illustration of the invention and are not intended to be limiting thereof.

### EXAMPLES

In the following examples all amounts are given in terms of parts by weight.

### Example 1

### Basic Moisturizer

The following ingredients were used in the following amounts:

| | |
|---|---|
| Lauroyl Lysine | 50 |
| Sodium Hydrogenated | 29 |
| Tallow Glutamate | |
| Trehalose | 10 |
| Acrylate/Alkyl Acrylate | 7 |
| Cross Polymer | |
| Arginine | 4 |

The ingredients are combined in a micro-pulverizer, to afford a powder. The powder may be diluted with a dilution factor of 5 wt % to 10 wt % of the powder concentrate with 90 wt % to 95 wt % of water at room temperature, with simple mixing, to give a moisturizer.

### Example 2

### Clarifer

The following ingredients were used in the following amounts:

| | |
|---|---|
| Lauroyl Lysine | 35 |
| Sodium Hydrogenated | 30 |
| Tallow Glutamate | |
| Acrylate/Alkyl Acrylate | 5 |
| Cross Polymer | |
| Pyrrolidone Carboxcylic Acid | 5 |
| Citric Acid | 25 |

The ingredients are combined in a micro-pulverizer, to afford a powder. The powder may be diluted with a dilution factor of 5 wt % to 10 wt % of the powder concentrate with 90 wt % to 95 wt % of water at room temperature, with simple mixing, to give a clarifier.

### Example 3

### Foundation Make Up

The following ingredients were used in the following amounts:

| | |
|---|---|
| Lauroyl Lysine | 46 |
| Sodium Hydrogenated | 25 |
| Tallow Glutamate | |
| Acrylate/Alkyl Acrylate | 4 |
| Cross Polymer | |
| Iron Oxides | 15 |
| Titanium dioxide | 10 |
| Talc | 10 |

The ingredients are combined in a micro-pulverizer, to afford a powder. The powder may be diluted with a dilution factor of 5 wt % to 10 wt % of the powder concentrate with 90 wt % to 95 wt % of water at room temperature, with simple mixing, to give a make up foundation.

### Example 4

### Sunscreen

The following ingredients were used in the following amounts:

| | |
|---|---|
| Lauroyl Lysine | 27 |
| Sodium Hydrogenated | 20 |
| Tallow Glutamate | |
| Trehalose | 5 |
| Acrylate/Alkyl Acrylate | 5 |
| Cross Polymer | |
| Arginine | 3 |
| Titanium Dioxide | 30 |
| Benzophenone | 10 |

The ingredients are combined in a micro-pulverizer, to afford a powder. The powder may be diluted with a dilution factor of 5 wt % to 10 wt % of the powder concentrate with 90 wt % to 95 wt % of water at room temperature, with simple mixing, to give a sunscreen.

As discussed above, the present powdered concentrates may be diluted at normal room temperatures to form moisturizers, creams, lotions, foundation make ups, cleansers and the like with simple mixing. The concentrate allows for single dose packaging, and obviates the necessity of anti-microbial preservation. Additionally, the powder concentrate form allows for low weight transport. Once diluted, the resulting composition(s) may be used in the same way that analogous conventional compositions are used.

The present invention overcomes these problems in a simple and elegant manner. It also allows the use of materials that would thicken the previous types of concentrates to an unusable viscosity. It also allows for the consumer to produce in home a fresh personal care product with simple mixing.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A composition, which comprises:
(a) a solid emollient or lipid; and
(b) a solid emulsifier.

2. The composition of claim 1, wherein said solid emollient is selected from the group consisting of long chain alcohols, long chain mono-, di-, and triglycerides, ethylene glycol diesters of long chain fatty acids, esters of a long chain alcohols with long chain fatty acids, salts of long chain fatty acids, fatty acid substituted amino acids, and mixtures thereof.

3. The composition of claim 1, wherein said solid emollient or lipid is selected from the group consisting of stearyl alcohol, cetearyl alcohol, cetyl alcohol, behenyl alcohol, glyceryl mono-, di, or tristearate, ethylene glycol distearate, cetyl palmitate, myristyl myristate, lauroyl lysine, aluminum stearate, zinc stearate, and mixtures thereof.

4. The composition of claim 1, wherein said solid emollient or lipid is selected from the group consisting of cetyl palmitate, myristyl myristate, behenyl alcohol, lauroyl lysine, and mixtures thereof.

5. The composition of claim 1, wherein said solid emollient or lipid has a solidification point greater than 35° C.

6. The composition of claim 1, wherein said solid emollient has a solidification point greater than 40° C.

7. The composition of claim 1, wherein said solid emollient or lipid is normally liquid at ambient temperatures and has been rendered in a solid form by mixing with a solid compound.

8. The composition of claim 7, wherein said solid compound is selected from the group consisting of urea and sodium carbonate.

9. The composition of claim 1, wherein said solid emulsifier is selected from the group consisting of salts of long chain fatty alcohol esters of sulfates; mono-, di-, and triesters of long chain fatty acids with sucrose; salts of long chain fatty acids; salts of long chain fatty acid esters and amides with glutamate; and mixtures thereof.

10. The composition of claim 1, wherein said solid emulsifier is selected from the group consisting of sodium lauryl sulfate, sodium cetearyl sulfate, sodium cetyl sulfate, sucrose stearate, sucrose distearate, sodium stearate, sodium tallowate, sodium hydrogenated tallowoyl glutamate, sodium stearoyl glutamate, disodium stearoyl glutamate, sodium lauroyl glutamate, sodium myristoyl glutamate, sodium cocoyl glutamate, potassium lauroyl glutamate, potassium myrsitoyl glutamate, and mixtures thereof.

11. The composition of claim 1, wherein said solid emulsifier is selected from the group consisting of sodium hydrogenated tallowoyl glutamate, sodium stearoyl glutamate, sodium lauroyl glutamate, sodium myristoyl glutamate, potassium lauroyl glutamate, potassium myristoyl glutamate, and mixtures thereof.

12. The composition of claim 1, wherein said solid emulsifier has a solidification point greater than 35° C.

13. The composition of claim 1, wherein said solid emulsifier has a solidification point greater than 40° C.

14. The composition of claim 1, wherein said solid emulsifier is normally liquid at ambient temperatures and has been rendered in a solid form by mixing with a solid compound.

15. The composition of claim 14, wherein said solid compound is selected from the group consisting of urea and sodium carbonate.

16. The powder concentrate of claim 1, comprising 10 to 90% by weight of said solid emollient or lipid and 10 to 90% by weight of said solid emulsifier, based on the total weight of said solid emollient or lipid and said solid emulsifier.

17. The powder concentrate of claim 1, further comprising an ingredient selected from the group consisting of sunscreens, antiperspirants, moisturizers, humectants, pigments, dyes, fragrance compounds, anti-bacterial agents, conditioning agents, vitamins, minerals, botanical extracts, exfoliants, protectants, calmatives, topical medicaments, and clarifying materials such as alpha hydroxy acids, beta hydroxy acids, thickeners, and enzymes.

18. The powder concentrate of claim 1, which is contained in a package in an amount of 0.5 to 50 grams of said powder concentrate.

19. A method of preparing a powder concentrate, said method comprising:
(1) mixing (a) a solid emollient or lipid and (b) a solid emulsifier.

20. The method of claim 19, wherein said solid emollient is selected from the group consisting of long chain alcohols, long chain mono-, di-, and triglycerides, ethylene glycol diesters of long chain fatty acids, esters of a long chain alcohols with long chain fatty acids, salts of long chain fatty acids, fatty acid substituted amino acids, and mixtures thereof.

21. The method of claim 19, wherein said solid emulsifier is selected from the group consisting of salts of long chain fatty alcohol esters of sulfates; mono-, di-, and triesters of long chain fatty acids with sucrose; salts of long chain fatty acids; salts of long chain fatty acid esters and amides with glutamate; and mixtures thereof.

22. The method of claim 19, wherein said solid emollient or lipid and said solid emulsifier are mixed in relative amounts of 10 to 90% by weight of said solid emollient or lipid and 10 to 90% by weight of said solid emulsifier, based on the total weight of said solid emollient or lipid and said solid emulsifier.

23. The method of claim 7, further comprising mixing an ingredient selected from the group consisting of sunscreens, anti-perspirants, moisturizers, humectants, pigments, dyes, fragrance compounds, anti-bacterial agents, conditioning agents, vitamins, minerals, botanical extracts, exfoliants, protectants, calmatives, topical medicaments, and clarifying materials such as alpha hydroxy acids, beta hydroxy acids, thickeners, and enzymes, with said solid emollient or lipid and said solid emulsifier.

24. A method for preparing an emulsion, said method comprising:
(1) mixing:
(a) an aqueous liquid, and
(b) a powder, said powder comprising:
(i) a solid emollient or a solid lipid; and
(ii) a solid emulsifier.

25. The method of claim 24, wherein said aqueous liquid is water.

26. The method of claim 24, wherein said solid emollient or lipid is selected from the group consisting of long chain alcohols, long chain mono-, di-, and triglycerides, ethylene glycol diesters of long chain fatty acids, esters of a long chain alcohols with long chain fatty acids, salts of long chain fatty acids, fatty acid substituted amino acids, and mixtures thereof.

27. The method of claim 24, wherein said solid emulsifier is selected from the group consisting of salts of long chain fatty alcohol esters of sulfates; mono-, di-, and triesters of long chain fatty acids with sucrose; salts of long chain fatty acids; salts of long chain fatty acid esters and amides with glutamate; and mixtures thereof.

28. The method of claim 24, wherein said powder concentrate comprises said solid emollient or lipid and said solid emulsifier in relative amounts of 10 to 90% by weight of said solid emollient or lipid and 10 to 90% by weight of said solid emulsifier, based on the total weight of said solid emollient or lipid and said solid emulsifier.

29. The method of claim 24, wherein said powder concentrate further comprises an ingredient selected from the group consisting of sunscreens, antiperspirants, moisturizers, humectants, pigments, dyes, fragrance compounds, anti-bacterial agents, conditioning agents, vitamins, minerals, botanical extracts, exfoliants, protectants, calmatives, topical medicaments, and clarifying materials such as alpha hydroxy acids, beta hydroxy acids, thickeners, and enzymes.

30. The method of claim 24, wherein said aqueous liquid and said powder concentrate are mixed in relative amounts of 0.05 to 1.0 parts by weight of aqueous liquid to 1 part by weight of said powder concentrate.

31. The method of claim 24, wherein said mixing is conducted at ambient temperature.

32. An emulsion prepared by a process, said process comprising:
(1) mixing:
(a) an aqueous liquid, and
(b) a powder, said powder comprising:
(i) a solid emollient or a solid lipid; and
(ii) a solid emulsifier.

33. The emulsion of claim 32, wherein said aqueous liquid is water.

34. The emulsion of claim 32, wherein said solid emollient or lipid is selected from the group consisting of long chain alcohols, long chain mono-, di-, and triglycerides, ethylene glycol diesters of long chain fatty acids, esters of a long chain alcohols with long chain fatty acids, salts of long chain fatty acids, fatty acid substituted amino acids, and mixtures thereof.

35. The emulsion of claim 32, wherein said solid emulsifier is selected from the group consisting of salts of long chain fatty alcohol esters of sulfates; mono-, di-, and triesters of long chain fatty acids with sucrose; salts of long chain fatty acids; salts of long chain fatty acid esters and amides with glutamate; and mixtures thereof.

36. The emulsion of claim 32, wherein said powder concentrate comprises said 15 solid emollient or lipid and said solid emulsifier in relative amounts of 10 to 90% by weight of said solid emollient or lipid and 10 to 90% by weight of said solid emulsifier, based on the total weight of said solid emollient or lipid and said solid emulsifier.

37. The emulsion of claim 32, wherein said powder concentrate further comprises an ingredient selected from the group consisting of sunscreens, anti-perspirants, moisturizers, humectants, pigments, dyes, fragrance compounds, anti-bacterial agents, conditioning agents, vitamins, minerals, botanical extracts, exfoliants, protectants, calmatives, topical medicaments, and clarifying materials such as alpha hydroxy acids, beta hydroxy acids, thickeners, and enzymes.

38. The emulsion of claim 32, wherein said aqueous liquid and said powder concentrate are mixed in relative amounts of 0.05 to 1.0 parts by weight of aqueous liquid to 1 part by weight of said powder concentrate.

39. The emulsion of claim 32, wherein said mixing is conducted at ambient temperature.
